(19) United States
(12) Patent Application Publication
(10) Pub. No.: US 2003/0145851 A1
(43) Pub. Date: Aug. 7, 2003

(54) Non-lethal gas apparatus to thwart hijacker

(76) Inventor: William C. Johnson, Urbana, OH (US)

Correspondence Address: William M. Selenke, 28 DeWitt Street, Cincinnati, OH, 45218, US

(21) Appl. No.: 10/061,378

(22) Filed: Feb. 1, 2002

(51) Int. Cl. A61M 16/00

(52) U.S. Cl. 128/203.12; 128/203.26

(57) ABSTRACT

Commercial aircraft need means of thwarting hijackers. Hijackers may kill or disable the pilot and fly the aircraft directly into a target building. A method of preventing such hijacking is to provide a non-lethal gas to incapacitate the hijackers such that they can be overpowered. This non-lethal gas should be placed to access the air duct of the ventilation system and placed in an area such as the cargo space that is not accessible to the passengers This system can be adapted to home use and for watercraft.

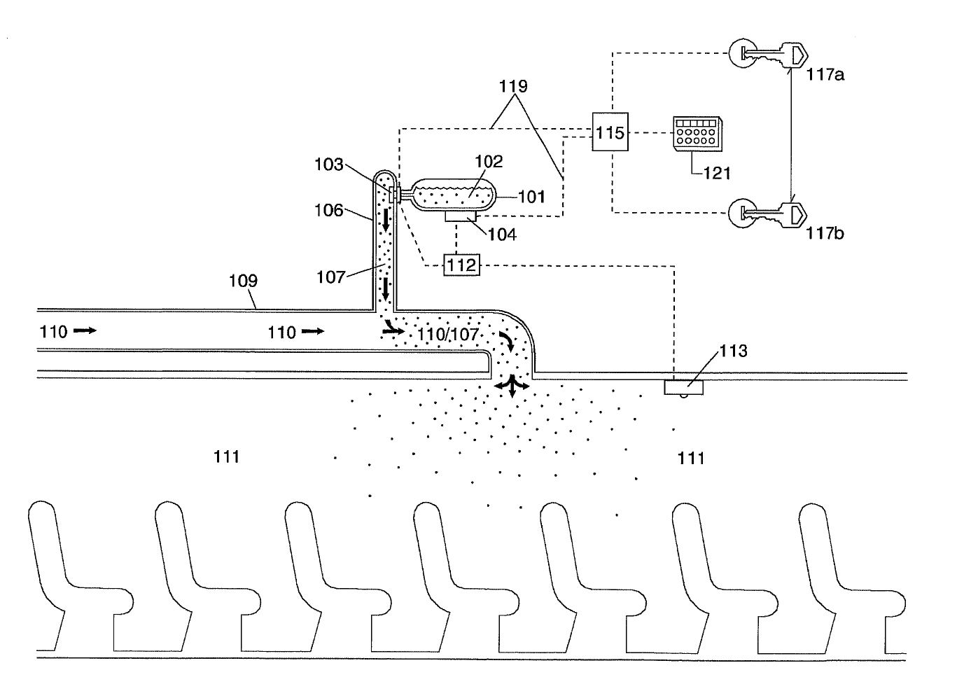

### FIELD OF INVENTION

The field of the invention is the use of non-lethal gases to incapacitate person or persons bent upon harm to aircraft or its passengers The system is especially useful in commercial aircraft hijack situations in which the hijacker wishes to kill or harm the pilot and take control of the aircraft with passengers to attempt a kamikaze attack on a target buildings. Likely target buildings are large buildings that have a strong emotional, symbolic presence.

### BACKGROUND OF INVENTION

From the early period of commercial aviation in the 1930's until the mid to late 1960's there were minimal restriction upon passengers and guests at airports. The passenger simply presented his ticket or money for a ticket and boarded the airplane with minimal difficulty. Friends or family members who wished to see the traveler off mixed freely with passengers.

This period of minimal restriction changed. In the late 1960's, a regular occurrence was that an armed passenger would hijack the airplane, typically to Cuba. The pilot followed the wishes of the hijacker. The passengers had a long flight and delay in Cuba. While this type of hijacking was a costly in terms of time and money, the passenger and airplane crew were not normally harmed.

However, because of the potential danger and the inconvenience of the regularly occurring hijackings, the FAA instituted a policy scanning passenger with metal detectors and the inspection of carry-on luggage with soft X-rays. These tactics prevented potential hijackers from carrying unauthorized guns into the body of the aircraft. This policy, coupled with the cooperation of Cuban authorities, reduced hijackings to near zero.

This effective program to prevent Cuban hijackings was shown to be quite inadequate to prevent dedicated zealots who were prepared to sacrifice their own lives as well as those of the passengers and crew. After receiving a certain basic flying proficiency, criminal zealots could direct a fully loaded aircraft into selected targets of symbolic and financial value.

At the present time, the old “rules” of hijacking are obsolete. Because there is a class of hijackers willing to learn how to fly a large commercial aircraft and guide that aircraft into a target, the current attitude towards hijackers is, “We or they.” That is anything the will control the hijackers and allow the safety of the pilot and crew is allowed.

### BRIEF DESCRIPTION OF FIGURE

The FIGURE provides a description of the device to lessen dangers to passengers in hijacking situations in aircraft or other passenger transport which is a non-lethal gas apparatus.

### DETAILED DESCRIPTION OF FIGURE

The FIGURE shows volatile somnolent substance container 101. Stored within container 101 is the volatile somnolent substance 102. The release of the volatile somnolent substance 102 is controlled by valve 103. A heater 104 to optimize the volatilization of the somnolent substance 102 is placed in proximity to the volatile somnolent substance container 101.

A tube 106 directs the flow of the in gaseous form 107 into the ventilation duct 109 which provides air conditioned breathing air 110 to the passengers compartment 111.

A monitor 113 in the passenger compartment 111 determines the level of the somnolent substance 102 and via feedback control electronics to the heater 104 and the valve 103.

The controls 115 for the activating means for the system is located in a place such that only the pilot or captain can activate it. The controls 115 through the connection means 119 can activate the container valve 103 and the heater 104. Heater 104 is one means of vaporizing the volatile somnolent substance 102. The connection means 119 between the controls 115 and the valve 103 and other components of activation system can be either electrical or mechanical.

The somnolent substance 102 apparatus must have a security activating system 115. Such a system would have activating means which would be is accessed through security devices such as a double key device 117A, 117B. Because of the seriousness of certain actions, these certain action requires two authorized people, each with a separate key simultaneously to turn the keys to activate the system. Thus, the “two key” system. A second of making secure the activation of the somnolent gases is a code which is processed through a alphanumeric code processing device 121. The code is known only to the pilot or other responsible person, but unknown to the potential hijacker or other unauthorized persons. The authorized person can “punch in” the correct alphanumeric code to activate the system.

There are any number of somnolent gases, each with advantages and disadvantages. The final choice of would depend upon the airlines or appropriate authorities. Such a class of gases would include the following: nitrous oxide, chloroform, halothane, fluoroxene, methoxyflurane and halopropane, enflurane, isoflurane, and trichloroethelene. These are likely useful agents. However, other agents could be used within scope and intent of the present invention.

The device would have a hardened construction that would require serious tools to open it. The container 102, the valve 103, the heater, the connection 106 to the ventilation duct 109, as well as the connection between the controls 115 and the valve is to be constructed within an enclosed hardened system made of hardened plastic or metal so that is essentially tamper proof As a further measure, the somnolent substance 102 apparatus ideally should be stored within the cargo space of the aircraft.

This system can be adapted to thwart criminals and terrorists in any closed spaces such as homes and ships or other watercraft as well as commerical buildings with air duct systems such as banks.

### SUMMARY OF INVENTION

A device to lessen dangers to passengers in hijacking situations in aircraft or other passenger transport includes an enclosed hardened system with an activating means; which is accessed through security devices. The enclosed system contains mechanical or electrical controls to activate a reservoir of volatile somnolent substance. The reservoir has a means to vaporize said volatile somnolent substance and the device releases the volatile somnolent substance into the craft's ventilating duct at a level to induce sleepiness in the passengers and hijackers. The parts of the devices that can be so isolated is physically in the cargo space of the aircraft and thus not accessible to unauthorized persons.

The hardened system is made of strengthened metal or reinforces plastics. A hand moved valve or an electrical or electronic activated valve will be used to release the gas. A two key system or an alphanumeric code pad process security device will be useful to activate the .hijacker prevention device. Somnolent compounds may includes the following: nitrous oxide, chloroform, halothane, fluoroxene, methoxyflurane and halopropane, enflurane, isoflurane, or trichloroethelene. Likewise, an electrical heater can be used to vaporize the volatile somnolent substance. The level of substances to induce sleepiness in the passengers and hijackers is monitored by a sensor and feed back mechanism.

I claim:

1) A device to lessen dangers to passengers in hijacking situations in aircraft or other passenger transport comprising:
an enclosed hardened system with an activating means;
said activating means is accessed through security devices;
said enclosed systems contains mechanical or electrical controls to activate a reservoir of volatile somnolent substance;
said reservoir has a means to vaporize said volatile somnolent substance
said device releases said volatile somnolent substance into the craft's ventilating duct;
said release of said volatile somnolent substance is at a level to induce sleepiness in the passengers and hijackers.

2) The device as in claim 1 wherein said hardened system is physically in the cargo space of said aircraft and thus not accessible to unauthorized persons.

3) The device as in claim 1 wherein said hardened system is made of strengthened metal.

4) The device as in claim 1 wherein said hardened system is made of reinforces plastics.

5) The device as in claim 1 wherein said activating means is a hand moved valve.

6) The device as in claim 1 wherein said activating means is an electrical or electronic activated valve.

7) The device as in claim 1 wherein said security device is a two key system.

8) The device as in claim 1 wherein said security device is alphanumeric code pad.

9) The device as in claim 1 wherein said somnolent substance is chosen from the class of compounds which includes the following: nitrous oxide, chloroform, halothane, fluoroxene, methoxyflurane and halopropane, enflurane, isoflurane, or trichloroethelene.

10) The device as in claim 1 wherein said means to vaporize said volatile somnolent substance is heat provides by an electrical heater.

11) The device as in claim 1 wherein said level of said volatile somnolent substance to induce sleepiness in the passengers and hijackers is monitored by a sensor and feed back mechanism.

12) The device as in claim 1 wherein said other passanger transport are ships.

13) The device as in claim 1 wherein said device is used for home protection.

14) The device as in claim 1 wherein said device is used for commerical buildings such as banks
